(19) United States
(12) Patent Application Publication
(10) Pub. No.: US 2007/0042026 A1
(43) Pub. Date: Feb. 22, 2007

(54) Prophylactic and therapeutic treatment of topical and transdermal drug-induced skin reactions

(76) Inventor: John J. Wille, Trenton, NJ (US)

Correspondence Address: LAW OFFICES OF NANCY A. BIRD, 231 WALTON AVENUE, SO. ORANGE, NJ, 07079, US

(21) Appl. No.: 11/083,178

(22) Filed: Mar. 17, 2005

(51) Int. Cl. A61F 13/02; A61K 36/898

(52) U.S. Cl. 424/448; 424/766

(57) ABSTRACT

Botanically derived anti-irritants for prophylactic and therapeutic treatment of adverse skin reactions from application of transdermal or topical drug delivery system, permits the effective administration of a drug from a delivery system in which the drug, of a component of the delivery system comprises a skin irritant; and the delivery systems formed thereby.

### FIELD OF THE INVENTION

This invention relates to the area of topical and transdermal drug delivery, either continuous or pulsatile and concerns the elimination of adverse skin reactions due to the use of skin irritating and skin sensitizing transdermally and topically administered drugs and/or system components.

### BACKGROUND OF THE INVENTION

### References Cited in the Following Specification

U.S. Patent Documents

Amkraut A and Shaw J. (1991a). U.S. Pat. No. 5,077,054. Prevention of contact allergy by co-administration of a coticosteroid.
Amkraut A and Shaw J (1991b). U.S. Pat. No. 5,000,596). Prevention of contact allergy by co-administration of a corticosteroid with a sensitizing drug.
Amkraut A. (−1991). U.S. Pat. No. 5,049,387. Inducing skin tolerance to a sensitizing drug.
Amkraut A, (1992). U.S. Pat. No. 5,118,509. Inducing skin tolerance to a sensitizing drug.
Cormiez M, Amkraut A, and Ledger P. (1995). U.S. Pat. No. 5,451,407. Reduction or prevention of skin irritation or sensitization during transdermal administration of an irritating or sensitizing drug.
Cormiez M, Ledger P, Johnson J, and Phipps J. (1995). WO 9,506,497. Reduction of skin irritation and resistance during elctrotransport.
Phipps J B. (1993). U.S. Pat. No. 5,221,254. Method for reducing sensitization in iontrophoretic drug delivery.
Cormiez M, Ledger P, Amkraut A, Marty J. (1989). U.S. Pat. No. 4,885,154. Method for reducing sensitization in transdermal drug delivery and means thereof.
Cormiez M, Ledger P, Amkraut A, and Marty J. U.S. Pat. No. 5,304,739. Methods of reducing sensitization in transdermal drug delivery and means thereof.
Ledger P W, Cormiez M, and Amkraut A. (1992a). U.S. Pat. No. 5,120,545. Reduction or prevention of sensitization to drugs.
Ledger P, Cormiez M, and Amkraut A. (1992b). U.S. Pat. No. 5,149,539. Reduction or prevention of sensitization to drugs.
Cormiez P M, Ledger P, and Amkraut A. (1992). U.S. Pat. No. 5,160,741. Reduction or prevention of skin irritation to drugs.
Cormiez M, Ledger P, and Amkraut A. (1992b). U.S. Pat. No. 5,130,139. Reduction or prevention of skin irritation to drugs.
Pandol, Stephen J. et al. US Patent Application 20040259816 Compositions comprising plant-derived polyphenolic compounds and inhibitors of reactive oxygen species and methods of using thereof.
Rudnic, Edward M. et al. US Patent Application 20040253249 Pulsatile transdermally administered antigens and adjuvants.
Hermelin, Victor M. US Patent Application 20040062802 Maximizing effectiveness of substances used to improve health and well being.
Strausak, S. et al. U.S. Pat. No. 5,370,635. Device for Delivering a Medicament.
Giannos, S. A. et al. U.S. Pat. No. 6,068,853. Temporally Controlled Drug Delivery systems.
Dinh, S. M. et al. U.S. Pat. No. 6,018,679 Iontophoretic transdermal delivery and control of adverse side-effects.
Hermelin, Victor M. U.S. Pat. No. 6,214,379 Maximizing effectiveness of substances used to improve health and well being.
Hermelin, Victor M. U.S. Pat. No. 5,945,123 Maximizing effectiveness of substances used to improve health and well being.
Creton, I. U.S. Patent Application No.: 20020182238A1. Fibers as anti-irritant agents.
Komeyev, A. Y. U.S. Pat. No. 6,576,269 (Jun. 10, 2003). Treating open skin lesions using extract of sea buckthorn
Perricone. U.S. Pat. No. 6,437,004 (Aug. 20, 2002). Treatment of skin damage using olive oil polyphenols.
Wilson, S. B. U.S. Patent Application No.: 20020165170A1. Method of attenuating reactions to skin irritants.
Wille, U.S. Pat. No. 6,670,395, 2003
Wille, U.S. Pat. No. 5,716,987, 1998
Wille and Kydonieus, U.S. Pat. No. 5,843,979, 1998
Wille, Kydonieus and Castellana, U.S. Pat. No. 5,618,557, 1997
Wille and Kydonieus, U.S. Pat. No. 5,686,100, 1997
Wille and Kydonieus, U.S. Pat. No. 5,912,010, 1999
Wille, Kydonieus and Castellana, WO Patent 9,718,782, 1997
Wille and Kydonieus, European Patent 5,612, 525, 1994
Wille et al., WO Patent 9,718,782, 1997
J J Wille. Provisional Patent Application No.: Novel Topical Delivery System for Plant Derived Anti-irritants (Jul. 4, 2003).
   Other Publications:
Bos, J. The Skin Immune System, CRC Press, Boca Ratan, Fla., 1989
Murphy M. and Carmichael A. J., Transdermal drug delivery systems and skin sensitivity reactions. Incidence and management. Am J Clin Dermatol. 2000 November-December; 1(6):361-8
Wille J J., Novel Pant-Derived Anti-Irritants. Abstract in: J. Cosmet. Sci., 54: 106-107, 2003
Kalish R, Wood J, Wille J, and Kydonieus A., Sensitization of mice to topically applied drugs: albuterol, chlorpheniramine, clonidine and nadolol. Contact Dermatitis 35:76-82, 1996.
Kalish R, Wood, J A, Kydonieus, A, and Wille, J J., Prevention of contact hypersensitivity to topically applied drugs by ethacrynic acid: potential application to transdermal drug delivery. J. Controll. Rel., 48:79-87, 1997
Wille J J, Kydonieus A., and Kalish R S., Inhibition of irritation and contact hypersensitivity by ethacrynic acid. Skin Pharm. Appl. Skin Physiol., 11(4-5):279-288, 1998
Wille J J, Kydonieus A, and Kalish R S., Several different ion channel modulators abrogate contact hypersensitivity in mice. Skin Pharm. Appl. Skin Physiol., 12(1-2):12-17, 1999a
Wille J J, Kydonieus A F, and Murphy G F., cis-Urocanic acid induces mast cell degranulation and release of preformed TNF-alpha: A possible mechanism linking UVB and cis-urocanic acid to immunosuppression of contact hypersensitivity. Skin Pharm. Appl. Skin Physiol., 12(1-2):18-27, 1999b
Wille J J, Kydonieus A, and Kalish R S., Inhibition of irritation and contact hypersensitivity by phenoxyacetic acid methyl ester. Skin Pharm. Appl. Skin Physiol., 13(2): 65-74, 2000.
J J Wille. Novel Topical Delivery System for Plant derived Hydrophobic Anti-Irritant Actives. Abstract presented at National Annual ACS Meeting NY, N.Y. (Sep. 17, 2003).
J J Wille. Plant-derived anti-irritants. In Closing with John Wille. Cosmetics & Toiletries Vol. 118(8): 128, 2003.
J J Wille. Thixogel: Novel Topical Delivery System for Hydrophobic Plant Actives. In: Personal Care Delivery Systems and Formulations Noyes Publication (in press).
J J Wille. Cutaneous delivery of Antioxidant Botanicals, 23^{rd} Annual Congress of IFSCC Abstract (October 2004, in press).

The transdermal and topical delivery of new drugs has been hindered due to the “Achille's heel” of transdermal drug delivery, i.e., the skin immune system (Bos, J. The Skin Immune System, CRC Press, Boca Ratan, Fla., 1989). The vast majority of drugs that could be delivered through the skin are unable to be approved by regulatory bodies due to safety issues concerning their skin irritation and or skin sensitization. These include drugs belonging to such groups as anti-addiction, anti-histamine, anti-asthmatic, cardiovascular, anti-inflammatory, anti-depressive, etc. This is unfortunate in that a patient compliant, single dose application lasting 7 days with elimination of adverse systemic side effects could all be improved through the transdermal drug delivery route. Only a dozen drugs have successfully entered the commercial market in transdermal delivery form in the past 20 years, and these are ones that proved not to have any adverse skin reactions. Allergic contact dermatitis may sometimes present after months or years of continuous exposure. In fact, the Clonidine TD patch was approved before it was discovered that 20% of patents became immune to it several months after continued transdermal use and displayed severe signs of allergic contact dermatitis (Kydonieus and Wille, 2000).

Tables 1 list examples of drugs, in a number of categories, unable to be approved by regulatory bodies for delivery through the skin due to safety issues concerning their skin irritation and or skin sensitization.

TABLE 1
Some Common Drugs That Are Either Topically
Irritating or Sensitizing

| Class of | Sensitizing | | Irritating |
|---|---|---|---|
| Drugs | Drugs | Class of Drugs | Drugs |
| Antiasthmatic | Albuterol | Ace inhibitor | Captopril |
| | | | Fosinopril |
| Antihypertensive | Clonidine | Antihypertensive | Prazosin |
| Antianxiety | Alprazolam | Antiseptic | Chlorhexidine |
| | Lorazepam | | |
| | Fluphenazine | | |
| Antihistamine | Clorpheniramine | Antiacne/anti- | Retinoic acid |
| | Diphenhydramine | wrinkling | α-Hydroxy |
| | | | acid |
| Beta blockers | Metaprolol | Cold medicine | Dimemorfan |
| | Nadolol | | |
| | Timolol | | |
| | | Enhancers | Propylene |
| | | | glycol |
| | | | Oleic acid |
| | | | Alcohol |
| | | Non-steroidal | Ketoprofen |
| | | Anti- | Diclofenac |
| | | inflammatory | Piroxicam |
| | | | Flurbiprofen |
| | | | Prostaglandin |

From: Kydonieus, Wille & Murphy, Biochemical Modulation of Skin Reactions CRC Press, Boca Ratan, Fl, pp.9-10, 2000

Potential allergens in transdermal delivery systems include the adhesive, the membrane, the solvent, the enhancer, and the active drug. The majority of reactions reported have been to the drug itself. Where the reaction has been shown to be to a component of the delivery system itself it may be possible, as an increasing number of products become available, to substitute another delivery system and continue use of the transdermal route. Conversely, patients who become sensitized to a component of the delivery system of one drug may experience reactions with transdermal delivery systems of other drugs. Contact dermatitis presents with irritation, erythema, edema, and occasionally vesiculation. The reaction is localized to the site of application of the current patch, but may also occur at the site of previous applications, reflecting residual antigen deposition. The onset of the time of reactions is very variable and may occur months after initiation of treatment. Deterioration of response to the drug has been reported as a presentation of allergic contact dermatitis (Murphy M. and Carmichael A. J., Transdermal drug delivery systems and skin sensitivity reactions. Incidence and management. Am J Clin Dermatol. 2000 November-December; 1(6):361-8).

In cases where contact allergic dermatitis has been demonstrated to the drug component of the TTS, patients have frequently been tolerant of subsequent oral challenges, but there have been reports of systemic sensitization also. The fact that patients are tolerant of subsequent oral challenge in most cases, reflects the role of local factors such as skin concentration, prolonged exposure and the local irritation in the expression of contact allergic dermatitis. However, in view of potential risk of a generalized reaction oralchallenges should be only performed where necessary and in a hospital environment—(Murphy and Carmichael, 2000, above). Several transdermal drug companies have realized these skin-related limitations and attempted to eliminate drug-induced contact hypersensitivity reactions due to transdermal drug delivery of sensitizing and irritating drugs. These attempts have been reviewed elsewhere: Wille and Kydonieus, (2000), and in U.S. Pat. Nos. 5,618,557; 5,686,100; 716,987; 584,979; 5,912,010; and 6,670,395.

Among the agents recently proposed for minimizing skin irritation due to skin irritants are fibers (Creton, I. (2002) U.S. Patent. Publication, 20020182238), and an immune suppressant such as a composition that blocks CD1d activation (Wilson, S. B (2002), U.S. Patent Publication, 20020165170) that blocks antigen presentation by skin located immune cells. Use of a histamine antagonist and or a TNF-alpha antagonist have also been proposed (Lacharriere, O. De; et al. (2001) in U.S. Publication.: 20010022978A1. Additional strategies to reduce skin irritation by various means have been proposed and investigated.

One such strategy is to provide “off” periods as an advantage over existing transdermal systems that currently provide constant delivery. These periods when less active ingredient is being delivered through the skin gives the skin where the patch is applied, an opportunity to “recover” thereby reducing skin irritation. Other benefits which may be realized by reducing skin irritation, are improved patient compliance to treatment through less irritation, greatly reduced side effects and greater drug efficacy. Devices, delivery mechanisms and applications are described in U.S. Pat. Nos. 5,370,635 and 6,068,853.

Another method, disclosed in Dinh, S. M. et al. U.S. Pat. No. 6,018,679 describes iontophoretically removing any compound that is capable of causing skin irritation or other harmful effects. More specifically, a bipolar iontophoretic transdermal delivery method is disclosed that includes a reversal phase for controlling the iontophoretic transdermal delivery of pharmaceutical compounds while reducing or eliminating skin irritation and terminating the pharmacological or toxicological effects in skin of drugs/cosmetics/active ingredients which form a drug depot in the skin after passive or iontophoretic application.

A truly effective strategy for elimination of transdermal drug-induced adverse skin irritation seeks to modulate checkpoints in the skin immune and skin irritant signal cascade. Earlier, Wille & Kydonieus (2000) reviewed the scientific and patent literature on anti-irritants and counter-sensitizers. The purpose of which, was to find new agents useful in prevention and treatment of contact dermatitis including both irritant and allergic contact dermatitis, due to dermatological and transdermal drugs. In a series of patents, it was shown that ion channel modulators and mast cell degranulating agents were effect anti-irritants and counter-sensitizers. Ethacrynic acid (Edecrin®, Merck) was effective in preventing contact sensitization due to the delivery to mouse skin of four sensitizing drugs: Clonidine, Chlorpheniramine, Albuterol, and Nadolol. Ethacrynic acid, a potassium ion channel blocker, was also effective in preventing skin irritation due to the topical application to mouse of 2,4-dinitro-chlorobenzene, arachidonic acid, phorbol myristic acid, trans-retinoic acid, and lactic acid. The calcium ion channel blockers Nifedipine and Verapamil were effective in minimize contact sensitization in mouse skin due to topical application of the sensitizing and transdermally delivered drug, Nadolol. Phenoxyacetic acid and its alkyl derivatives, a non-drug analog of the diuretic, ethacrynic acid, was shown to prevent contact sensitization due to application to mouse skin of the sensitizing hair dye, para-phenylenediamine and to block skin irritation due a panel of known skin irritants, including anionic surfactants such as sodium lauryl sulfate. Finally, agents, which induce mast cell degranulation such as cis-urocanic acid and capsaicin were reported to prevent contact sensitization in a mouse skin model. [Wille, U.S. Pat. No. 6,670,395; Wille and Kydonieus, U.S. Pat. No. 5,912,010, Wille, U.S. Pat. No. 5,716,987, Wille and Kydonieus, U.S. Pat. No. 5,843,979, Wille, Kydonieus and Castellan, U.S. Pat. No. 5,618,557, Wille and Kydonieus, U.S. Pat. No. 5,686,100, Wille, Kydonieus and Castellana, WO Patent 9,718,782, Wille and Kydonieus, European Patent 5,612,525; and in reports (Kalish R, Wood J, Wille J, and Kydonieus A, 1996; Kalish R, Wood J A, Kydonieus A, and Wille J J., 1997: Wille, J J, Kydonieus A, and Kalish R S, 1998; Wille, J J., Kydonieus, A., and Kalish, R S., 1999a; Wille, J J, Kydonieus, A F., and Murphy, G F., 1999b; Wille, J J., Kydonieus, A., and Kalish, RS., 2000; Wille and Kydonius, 2001.]

Efforts to overcome contact dermatitis have led to strategies to incorporate anti-irritants and counter-sensitizers in drug patches. Corticosteroids have been claimed in a series of patents by ALZA [Amkraut A and Shaw J., U.S. Pat. No. 5,077,054; Amkraut A and Shaw J., U.S. Pat. No. 5,000,596; Amkraut A., U.S. Pat. No. 5,049,387; Amkraut A,. U.S. Pat. No. 5,118,509; Cormiez M, Amkraut A, and Ledger P. U.S. Pat. No. 5,451,407; Cormiez M, Ledger P, Johnson J, and Phipps J. (1995). WO 9,506,497; Phipps J B. U.S. Pat. No. 5,221,254; Cormiez, M., Ledger P, Amkraut A, Marty J. U.S. Pat. No. 4,885,154; Cormiez M, Ledger P, Amraut A, and Marty J. U.S. Pat. No. 5,304,739; Ledger PW, Cdortmiez M, and Amkraut A). U.S. Pat. No. 5,120,545; Ledger P, Cormiez M, and Amkraut A. U.S. Pat. No. 5,149,539; Cormiez P M, Ledger P, and Amkraut A. U.S. Pat. No. 5,160,741; Cormiez M, Ledger P, and Amkraut A. U.S. Pat. No. 5,130,139]. Unfortunately, steroids have adverse effects on skin when they are topically applied over an extended period as would be required for chronic user in the treatment of systemic disease using transdermal drugs that are skin irritating or skin sensitizing. Similarly, the use of drugs in a transdermal patch other than the drug to be delivered systemically poses major safety issues. For this reason, many useful immune suppressive drugs cannot be used in the transdermal context.

Another approach that avoids the consequences of employing drugs with prescription indication different from the drug to be administered transdermally is to choose natural products that only affect the skin. This is the approach of the invention of the present application.

Natural products and plant extracts have been the focus of recent interest as emollients and anti-irritants. The following are examples of natural products and plant extracts which have prevent, reduce, or eliminate skin irritation. Castro J (1995) in U.S. Pat. No. 5,393,526 discloses Rosmarinic acid (5%), derived from Sage plant was able to reduce by over three-fold the irritating action of alpha-hydroxy acids (lactic and glycolic acids). Pretreatment one-half hour prior to application of cosmetic formulation containing known skin irritants, para-aminobenzoic acid and balsam of Peru with extracts the Cola nitida plant have been disclosed to prevent skin irritation, as described in European Patent Application 0,354,554A2. Oil from Yerba plants have also been claimed to eliminate irritation and sensitization that accompanies topical, tranmucosal and transdermal delivery of dihydroergotamine mesylate, acetominophen, oxymetazoline, diphenhydramine, nystatin, clindamycin, and para-aminobenzoic acid. See World Patent Application WO 9,114,441. Oils of chamomile, containing chamazulene isolated from yarrow, chamomile and wormwood, were disclosed to be good antipuretics when co-administered in transdermal Nicotine patches (U.S. Pat. No. 4,908,213).

Depletion of antioxidants is known to cause oxidative damage to human skin (Podda et al). Topical replacement of skin anti-oxidants may help to alleviate irritation due to ultraviolet radiation and ozone exposure. As discussed above, flavonoids are known to be potent anti-oxidants, however, they require stabilization against oxidation by addition of co-reductants such as Vitamin E (alpha-tocopherol) or Vitamin C (Ascorbic Acid). No mechanism exists to reduce oxidized Vitamin E since there is no Ascorbic acid in the upper layers of the epidermis (stratum corneum). Lazendorfer et al., in U.S. Pat. No. 6,423,747 discloses cosmetic and dermatological preparations with favonoids having anti-oxidant properties. Examples disclosed therein mention standard water-in-oil and oil in water formulations without providing any evidence of efficacy in these formulations.

Of particular importance to the category of polyphenols and flavonoids is the demonstration (Wille, 2003) that the mechanism of action for many plant-derived anti-irritants is their inhibition of protein tyrosine kinases associated with growth factor receptor stimulated autocrine control of cell proliferation that is the hallmark of many useful skin products that cause skin irritation, i.e., retinoic acid. The use of flavonoids as anti-irritants are among the plant-derived anti-irritants that are readily formulated in the novel hydrophobic delivery system claimed by Wille (U.S. Patent Appl No. 10/873,590). They include many plants and herbs are rich in flavonoids as well as flavonoids present in Spanish Honeybee pollen. For example, rutin, quercetin, myricetin, and trans-cinnamic acid; all were present at >350 mg/100 g. Recently, it was reported (Bonina et al, 2002), that Kaempferol is the major flavonoid derived from lyophilized extracts of the flowering buds of capers (Capparis spinosa L). This material was shown to have both anti-oxidant and photo-protective effects in human skin.

Antioxidants and free radical scavengers have been employed in many patented formulations for eliminating or minimizing irritation and contact sensitization reactions. Inhibitors of the metabolites of the arachidonic acid cascade known to be involved in the irritant mechanism of skin have been claimed in European Patent EP 0,314,528A1. Among the designated anti-irritants claimed were Vitamin E, BHT, para-tertiary butyl catechol, hydroquinone, benzoquinone, N,N-diethylhydroxyamine, and nordihydroguaiareic acid.

Vitamin C (ascorbic acid), a water soluble antioxidant, was disclosed in U.S. Pat. No. 5,516,793 to be effective in decreasing skin irritation caused by topical application of such ingredients as: α-hydroxy acids, benzoyl peroxide, retinal, retinoic acid, quaternary ammonium lactates, and salicylic acid. Vitamin E (alpha-tocopherol) is disclosed in U.S. Pat. No. 5,545,407 to reduce skin irritation caused by actives in dermatological preparations containing benzoyl peroxide, and in U.S. Pat. No. 5,252,604 it was disclosed that topical alpha-tocopherol reduced skin irritation due to repeated doses of retinoic acid. Another antioxidant panthenol and its derivatives pantothenic acid, pantethine and pantetheine have been claimed as anti-irritants for formulations containing up to 20% benzoyl peroxide.

The role of antioxidants in protecting the skin from harmful solar exposure, and photoaging is well known. In their book, “Oxidants and Antioxidants in Cutaneous Biology, Thiele and Elsner (2001) have assembled a comprehensive review of free radical chemistry in the skin and the antioxidant network of defense in the stratum corneum. Among the antioxidants discussed for protection of skin are Vitamins E and Vitamin C, green tea polyphenols, resveratol, curcumin, silymarin, ginger, and diallyl sulfide, all of which afford some protection against the development of skin cancer. In addition, the role of carotenoids (lycopenes, luein and alpha, and beta-carotene) as dietary supplements in chemoprevention of cancer are reviewed. Anti-oxidants with protective effect of topical anti-oxidants against solar radiation include: Vitamin E and Vitamin C. Other reported antioxidants that efficiently reduce photodamage include the thiol, N-acetylcysteine and α-lipoic acid, which may prevent oxidative stress in skin. In addition, plant-derived flavonoids (apigerneic genistein, catechin, epicatechin, _-glycosylrutin and silymarin) are polyphenols with good antioxidant activity.

Other botanically-derived actives with potential anti-irritant activity are the catechins and polyphenols; e.g., green tea leaves, and grape seed oil extracts. Additional antioxidants derived from botanicals are Bis-Abolol, Epigallocatechine, Epigallocatechinegallate Rutin, Quercetin, Hesperidin, Diomine, Mangiferin, Mangostin, Cyanidin chloride, Astaxanthin, Xanthophylls, Lycopene, Reversatrol, Tetrahydrodiferuloylmethane, Rosmarinic acid, Hypericin, Ellagic acid, Chlorogenic acid, Oleeuropein, Thiotic acid, Glutathione, and Andrographolide (Gupta, 2001). Few of these have been rigorously shown to eliminate adverse skin reactions due to topical drug delivery.

In addition, a suitable delivery system is required for the delivery of plant-derived extracts enriched in polyphenols. Polyphenols possess well-known anti-irritants and antioxidant properties. Some polyphenol containing plant extracts that could benefit by formulation in the skin care delivery system include: Grape seed, Honeysuckle, Cranberry and Green Tea (Camellia sinensis leaves). These plant materials are also rich in flavonoids (Quercetin and Apigenin), chlorogenic acid, and epigallocatechingallate and caffeine.

Polyphenols and antioxidants have been used to prevent or treat diseases. Pandol, S. J. et al. (2004) US Patent Publication 20040259816 describes methods for treating, preventing, or inhibiting diseases and disorders associated with NF-.kappa.B activation including proliferative diseases such as cancer and inflammatory diseases such as pancreatitis in a subject which comprises administering at least one polyphenolic compound and/or at least one inhibitor of reactive oxygen species to the subject.

It is therefore an object of the invention to provide a method to prevent, inhibit, or modulate adverse skin reactions and sensitization due to the transdermal or topical delivery of pharmaceutically active agents and/or cosmetic active agents.

It is also an object of the present invention to provide a method to prevent, inhibit, or modulate adverse skin reactions and sensitization due to the transdermal or topical delivery system components.

It is another object of the present invention to utilize botanically-derived actives such as polyphenols, flavonoids and antioxidants as the anti-irritant and/or anti sensitizer.

It is further an object of the present invention to provide a method using the anti-irritant and/or anti sensitizer in combination with a continuous topical and/or transdermal dug delivery system.

It is still further an object of the present invention to provide a method using the anti-irritant and/or anti sensitizer in combination with a pulsatile topical and/or transdermal dug delivery system. Such a method would provide improved efficacy of an active agent by controlling the temporal release of the active agent.

It is even further an object of the invention to provide an anti-irritant and/or anti sensitizer with an active agent that is being delivered in a temporal manner in a synchronous pattern with rhythmic body cycles especially for the treatment of diseases associated with disorders of circadian rhythm or other chronobiological malady.

### SUMMARY OF THE INVENTION

This invention concerns a method for the elimination of adverse skin reactions due to the use of skin irritating and skin sensitizing transdermally and topically administered drugs and/or delivery system components, whether through continuous or pulsatile delivery; and the delivery systems constructed utilizing the method. Also disclosed are new, anti-irritant uses of compounds, providing prophylactic and therapeutic treatment of transdermal and topical drug-induced contact dermatitis. In corporation of these anti-irritants in transdermal and topical drug delivery systems may be used to treat, prevent, eliminate, reduce, inhibit or modulate adverse skin reactions

Examples of pharmaceutically active agents which may benefit from transdermal or topical delivery are adrenergics, antiasthmatics, antiarrhythmics, anticancer drugs, anti-AIDS medications, anti-parkinsonian drugs, anti-anginals, Alzheimer's medications, somatomedins, cardiovascular and hypertensive drugs, diabetes medications, anti-viral agents, antisense peptides, anti-ulcer medications, sleep medications, PMS therapeutics, analgesics, endocrine/reproductive therapeutics, birth control medicaments, general hormone replacement therapeutics, pain medications, NSAIDs, epileptic medications, migraine headache medications, stroke medications, antibiotics, immunizations, addiction treatments, anxiolytics, anti-inflammatories, chemical or biological warfare agents and diagnostic and contrast media. The components of a traditional transdermal drug delivery system may include the membrane, the solvent, the agent used to enhance penetration of the pharmaceutically active agent, and the pharmaceutically active agent.

The general class of agents proposed for accomplishing this purpose are potent anti-oxidant compounds that possess anti-irritant and free radical scavenging properties. Polyphenolic compounds may also be used to accomplish this purpose. The polyphenolic compounds may be derived or isolated from plants. In some embodiments, the polyphenolic compound is a flavonoid. In other embodiments, the polyphenolic compound is a non-flavonoid.

The potent antioxidants that are useful for prophylactic and therapeutic treatment of adverse skin reactions due to transdermal drug delivery include several hydroxybenzoic acids, their congeners, and their esters. These include but are not limited to caffeic acid (3,4-dihydroxy cinnamic acid, [331-39-5]), gallic acid (3,4,5-trihydroxybenzoic acid, [149-91-7]), two naturally occurring phenolic compounds, and their esters including but not limited to caffeic acid phenethyl ester, phenethyl-3-methylcaffeate, ferulic acid (3-methyl ether of caffeic acid [1135-24-6]), and chlorogenic acid [327-97-9], an analog of caffeic acid.

The possible mechanism of action of caffeates include antioxidant or electrophilic trapping as modulators of arachidonic acid metabolism cascade pathways, cell protein kinase inhibition, and inhibition of carcinogenesis (Newmark, H, L. Dietary Phytochem. In Cancer Prevention and Treatment, AICR, NY., pp. 25-34, 1996).

We also claim curcumin, a compound that is structurally related to caffeic acid, as an agent for prophylactic and therapeutic treatment of skin irritation from topical or transdermal drug delivery systems. Curcumin [458-37-7] inhibits lipoxygeneases and cyclooxygeneases and is an antioxidant with anti-inflammatory properties (Huang M T, Lysz, T., Ferraro, T., Abidi, T. F., Laskin, J. D., Conney, A. H. Cancer Res., 51: 813-9, 1991).

In addition the use of flavonoid antioxidants found in extracts from green teas are covered as agents for prophylactic and therapeutic treatment of skin irritation from topical or transdermal drug delivery systems. The flavonoids occur as glycosides and consist of flavones, flavonols, isoflavones, and flavonones. These include: catachin [154-23-4], chalcone [614-47-1], epicatechin [490-46-0] epicatechin gallate [1257-08-5], epigallocatechin [970-74-1], epigallocatechin gallate [989-51-5] and thgeir congeners and esters. Additional flavonoids that are useful antioxidant to treat transdermal drug-induced contact dermatitis include: chalcone [614-47-1], quercetin [117-39-5], genestein[446-72-0] and daidzein [486-66-8] and their t congeners and esters. Quercetin inhibits lipoxygeneases (Nakadate, T., Aizu, E, Yamamoto, S, Kato, R. Prostaglandins, 30: 357-68, 1985) and has good anti-inflammatory activity. The dried powders of cocoa bean plants, like green tea leaves, contain both methylxanthines and polyphenols. Again, extracts of cocoa powder are disclosed here to be a rich source of anti-oxidants for use in the method and product of the present invention.

Examples of the polyphenolic compound useful in the present invention include flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol

### DETAILED DESCRIPTION OF THE INVENTION

### Example 1

### Antioxidant Activity of Hydroxybenzoic acids

Anti-Oxidant Assay:

Solutions of test compounds were assayed for their antioxidant activity by the free-radical scavenging method that uses diphenylpicryl hydrazine (DPPH*) reagent as described previously (Bonina et al, 2003). In order to standardize the activity, we defined for each compound, an EC₅₀ value as the concentration that lowers the zero time optical absorbance of DPPH at 595 nm by 50 percent measured after 30 minutes of incubation at 25° C.

Antioxidant Activity of Test Compounds:

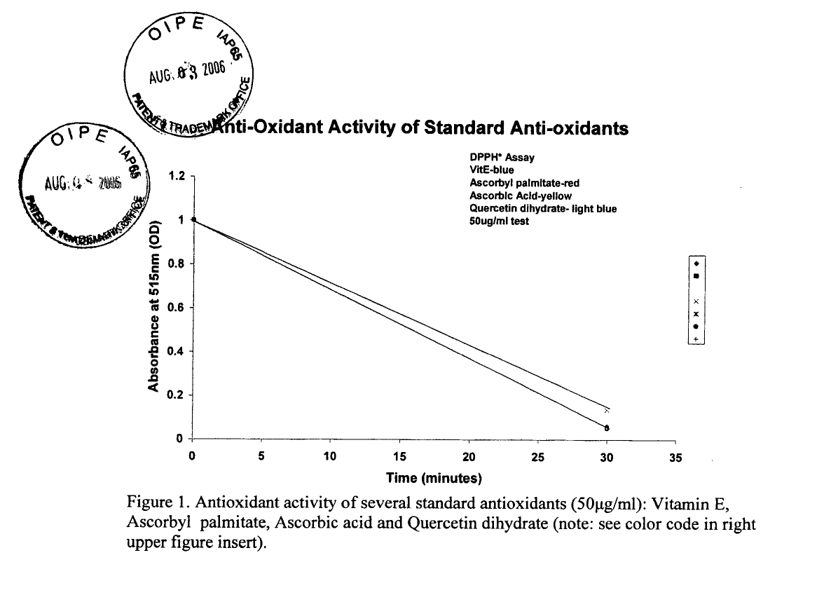

FIG. 1 shows a typical plot of antioxidant activity for several standard antioxidants as assayed by the DPPH* method. The molar activities of ascorbic acid, ascorbyl palmitate and vitamin E were calculated as 26 μM, 30 μM and 46 μM, respectively. Indole acetic acid, a weak free-radical scavenger and plant hormone, had a molar activity of 190 μM. Finally a commercially purchased flavonoid, quercetin dihydrate, had an intermediate molar activity of 86 μM.

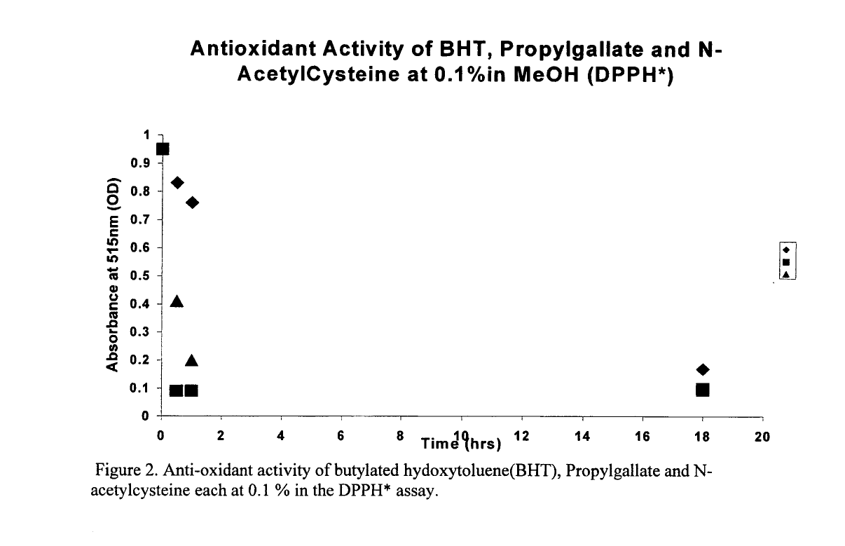

FIG. 2. presents results of DPPH* assay showing the anti-oxidant activities of butylated hydroxytoluene (0.1% BHT), propylgallate 0.1% PG), and N-acetylcysteine (0.1% N-AcC).

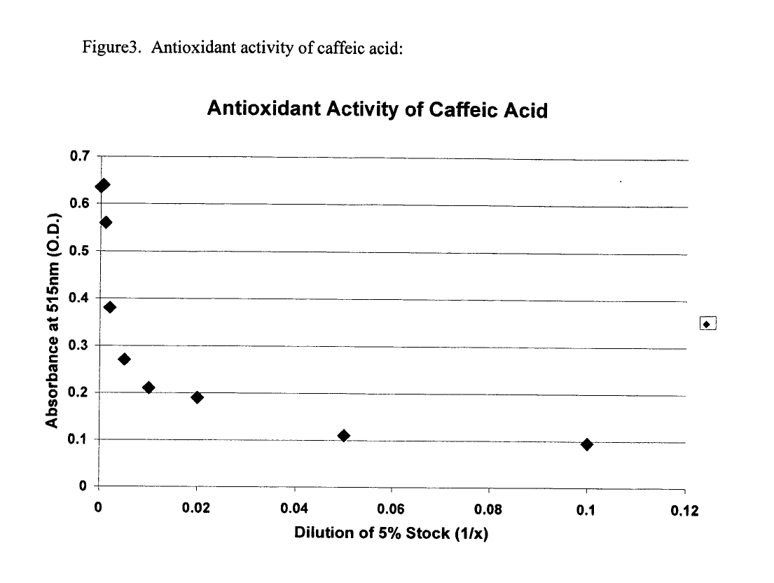

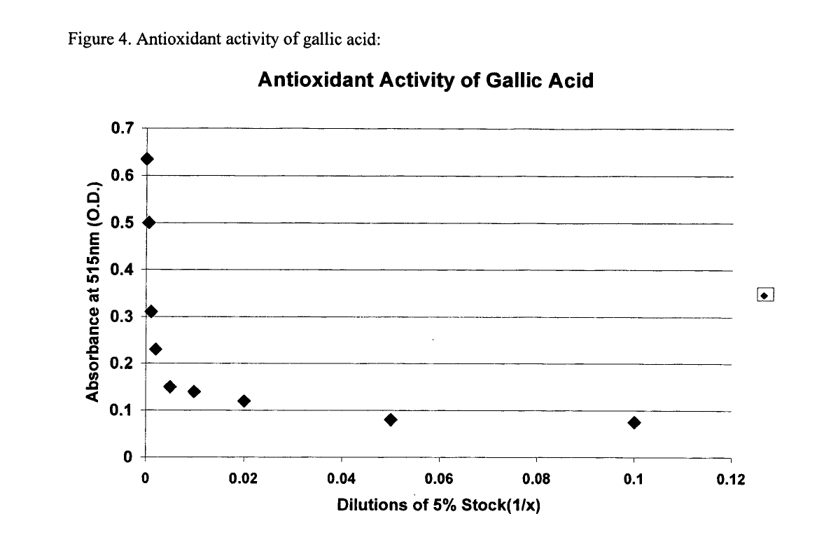

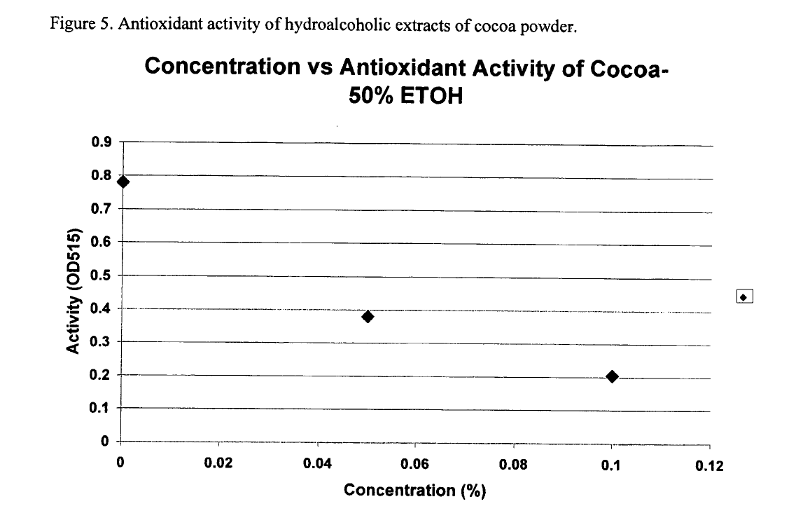

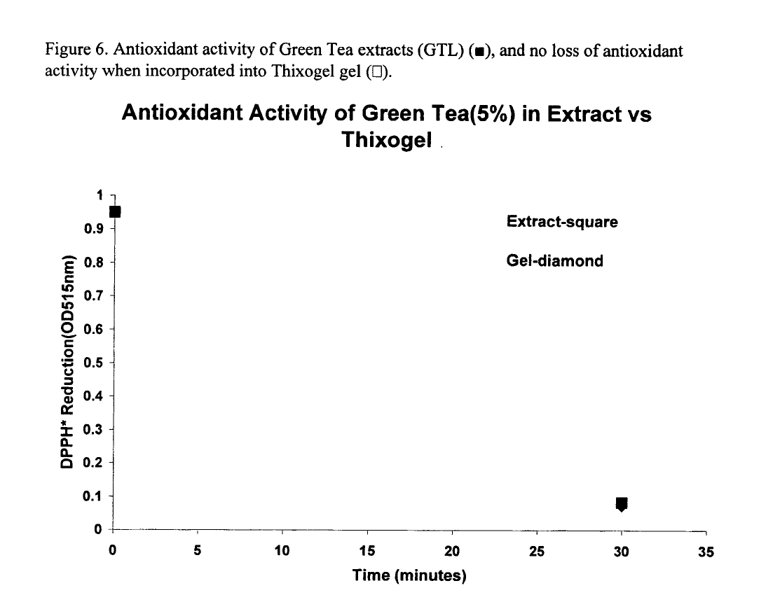

FIGS. 3 and 4 shows the results of assaying by the DPPH method the antioxidant activity of caffeic acid and gallic acid, respectively. We have also assayed hydroalcoholic extracts for their antioxidant activity. FIGS. 5 and 6 show the antioxidant activity of green tea leaves (GTL) and cocoa powder, respectively.

### Example 2

### Biochemical Assay for Detection of Anti-Irritant Activity

Since retinoids irritate skin leading to epidermal hyperplasia, we have employed an in vitro cell culture method to detect antioxidants that act to inhibit retinoid-stimulated autocrine growth of human keratinocytes.

Method: An immortalized line of human epidermal kertatinocytes, HaCat keratinocytes, can be cultured in a serum-free culture medium. Sterile Petri dishes (35 mm²) are seeded at 5,000 cells per cm² and placed in a humidifed CO₂ incubator at 37° C. for 3-5 days or until the culture reaches about 30% confluent monolayer growth. The dishes are washed once with ice-cold serum-free media lacking EGF and insulin, and refed 2.5 ml of serum-free culture medium containing 5 ug/ml insulin and retinyl acetate (RA, 3×10⁻⁸ M). Duplicate control dishes are fixed and stained with 0.2% crystal violet to record the amount of clonal growth prior to refeeding with fresh RA-containing medium. Test dishes refed RA and insulin are split into three groups in duplicate. Group A is refed on the RA plus insulin medium. Group B is refed RA plus insulin medium and a TRK inhibitor (PD 153035), and Group C is refed medium containing RA plus insulin and from 0.1 to 5% of a hydroalcoholic botanical extract. All dishes are adjusted to have the same final concentration of alcohol (1%). All dishes are placed back in the incubator for 2 and 4 days. They are fixed and stained with 0.2% crystal violet. The stained dishes are photographed for comparison of results.

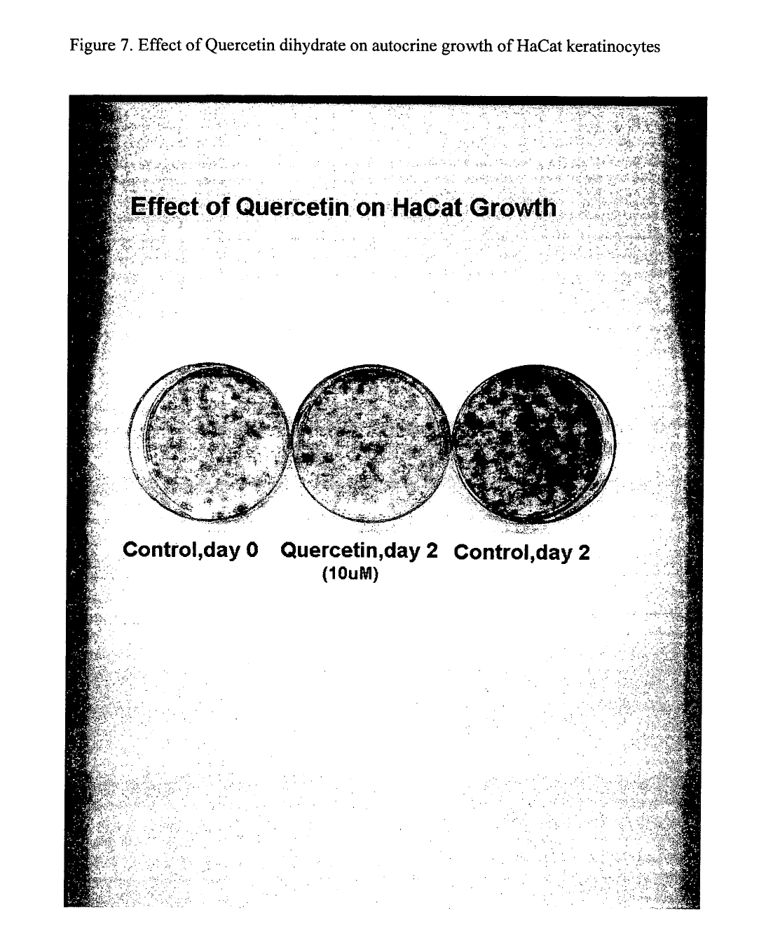

As a proof of principle the effect of Quercetin dihydrate (10 μM) a known inhibitor of growth factor receptor TRK on HaCat clonal growth was compared with growth of HaCat cells grown without Quercetin dihydrate, and both cultured under retinoid-stimulated autocrine growth conditions. As predicted, FIG. 7 shows that 48 hours after treatment with Quercetin dihydrate clonal growth was completely inhibited relative to the untreated control.

### Example 3

### In Vivo Test for Anti-Irritant Activity of Hydroxybenzoic Acid Compounds

Anti-Irritant Activity of Gallic Acid and Green Tea Extracts

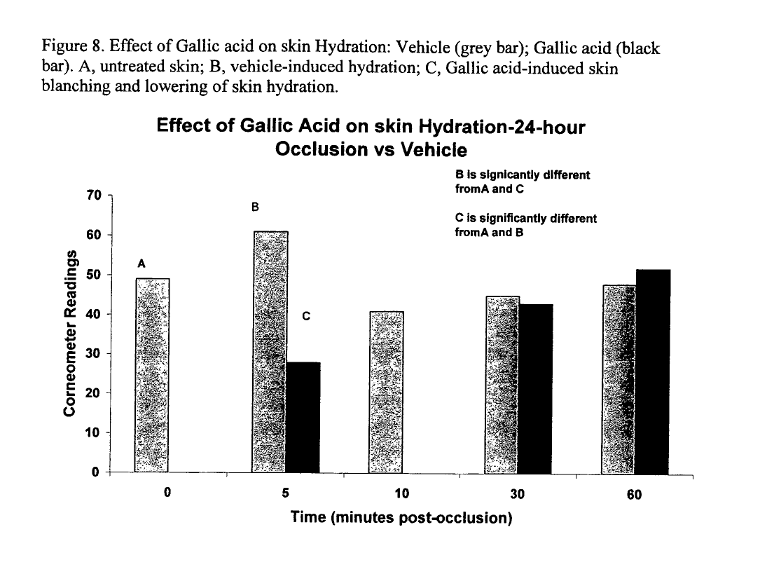

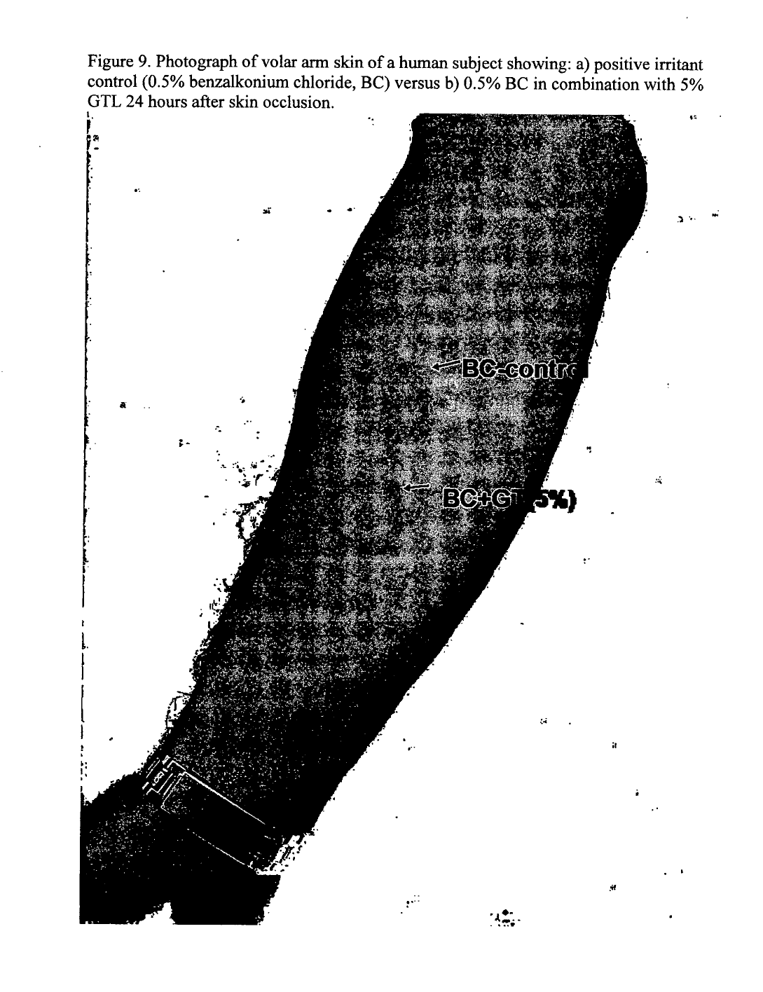

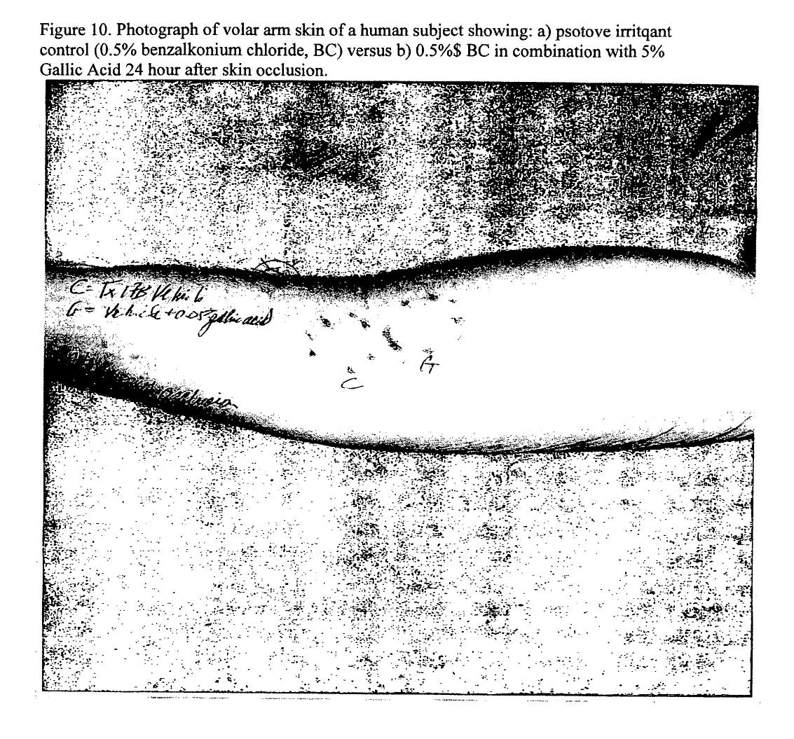

Occlusive patch testing was conducted on extracts prepared by starch gel encapsulation in a oil-in water emulsion system previously described (Wille, 2003). FIG. 8 presents results showing that a 5% Green Tea Leaf extract in vehicle gel elevated skin hydration following 24-hour occlusion when co-administered in the carrier gel with an irritating amount of benzalkonium chloride (0.5%). FIG. 9 presents results showing that gallic acid is not irritating to skin and does not alter skin hydration profile after 24 hours of occlusion and FIG. 10 shows the anti-irritant effect Gallic acid on benzalkonjium chloride induced skin irritation under 24 hour occlusion.

Anti-irritant assays: All carrier system gels were prepared with 0.5% benzalkonium chloride, a mild irritant. To test for anti-irritancy, the irritant-containing carrier gels were also loaded with the test botanical extracts (experimentals). The control and test gels were deposited (100 microliters) on 10 mm square circular filter paper discs and placed in side of Finn chambers (20 mm square), which were then applied to the volar arm skin of volunteers. The chambers were affixed to skin with non-allergic adhesive tape and left in place for 24 hours. Upon termination of the treatments, the chambers were removed and the skin gently wiped clean with moistened cotton swabs. The exposed skin was first examined for signs of erythema (redness) and induration (swelling), and skin sites photographed. The exposed skin sites were probed for skin capacitance (skin moisture levels) using a Corneometer instrument (Courage & Khazaka, Koln, Germany).

In summary, the results of our studies have demonstrated that hydroxybenzoic acids, flavonoids, and catechins from green tea leaves possess both potent anti-oxidant activities and either directly through skin patch testing, or through detection of surrogate biochemical end points for irritation are effective in eliminating skin irritation.

There has thus been shown and described novel botanically derived anti-irritants for prophylactic and therapeutic treatment of adverse skin reactions from application of transdermal or topical drug delivery system, which permit the effective administration of a drug from a delivery system in which the drug, of a component of the delivery system comprises a skin irritant; and the delivery systems formed thereby, which fulfills all the objects and advantages sought therefore. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

1. A method of treating, preventing, inhibiting, or modulating adverse skin reactions, irritation or sensitization produced by the components of a transdermal drug delivery system for administration of pharmaceutically active agents, by co-administration of 0.1% to 5% of one or more anti-oxidant, botanical agents derived from natural products and plant extracts.

2. The method of claim 1, wherein the antioxidants botanical agents derived from natural plant sources, are polyphenols.

3. The method of claim 2, wherein the polyphenols are selected from the group consisting of: hydroxybenzoic acids, their congeners, and their esters, ferulic acid and chlorogenic acid and curcumin; green tea extracts; chalcone [61447-1], quercetin [117-39-5], genestein[446-72-0] and daidzein [486-66-8] and their congeners and esters, and the powder of cocoa bean plants.

4. The method of claim 1, wherein the antioxidants botanical agents derived from natural plant sources, are phenols.

5. The method of claim 4, wherein the phenols are selected from the group consisting of: flavones, flavonols, isoflavones, and flavonones.

6. The method of claim 4, wherein the phenols are selected from the group consisting of: catachin [154-23-4*], chalcone [614-47-1], epicatechin [490-46-0] epicatechin gallate [1257-08-5], epigallocatechin [970-74-1], epigallocatechin gallate [989-51-5] and their congeners and esters.

7. The method of claim 3, wherein the hydroxybenzoic acids, their congeners, and their esters are selected from the group consisting of: caffeic acid, gallic acid, caffeic acid phenethyl ester, and phenethyl-3-methylcaffeate.

8. The method of claim 3, wherein the agents derived from dried powder of cocoa bean plants further comprise methylxanthines.

9. The method of claim 3 wherein the green tea extract is selected from the group consisting of: catechins including but not limited to catachin [154-234], chalcone [61447-1], epicatechin [49046-0] epicatechin gallate [1257-08-5], epigallocatechin [970-74-1], epigallocatechin gallate [989-51-5] and other antioxidants found in extracts from green teas.

10. The method of claim 2, wherein the polyphenolic compound is selected from the group consisting of flavenoids, anthrocyanins, anthrocyanidins, isoflavones, catechins, epigallocatechin gallate, gallic acid, chlorgenic acid, curcumin, kaempferol, quercetin, isoquercitrin, myricetin, rutin, pelargonidin, cyanidin, delphinidin, peonidin, malvidin, malvin, oenin, cyanidin, kuromanin, diadzein, daidzin, genitein, genistin, tannic acid, caffeic acid, ferulic acid and traxol.

11. The method of claim 2 wherein the polyphenolic compound is selected from the group consisting of: quercetin dihydrate [117-39-5], rutin, genestein [446-72-0], trans-resveratrol and daidzein [486-66-8].

12. The method of claim 1 wherein said pharmaceutically active agents are selected from the group consisting of adrenergics, antiasthmatics, antiarrhythmics, anticancer drugs, anti-AIDS medications, anti-parkinsonian drugs, anti-anginals, Alzheimer's medications, somatomedins, cardiovascular and hypertensive drugs, diabetes medications, anti-viral agents, antisense peptides, anti-ulcer medications, sleep medications, PMS therapeutics, analgesics, endocrine/reproductive therapeutics, birth control medicaments, general hormone replacement therapeutics, pain medications, NSAIDs, epileptic medications, migraine headache medications, stroke medications, antibiotics, immunizations, addiction treatments, anxiolytics, anti-inflammatories, chemical or biological warfare agents and diagnostic and contrast media.

13. The drug delivery method of claim 1, wherein the pharmaceutically active agent is administered at least once and may be administered as immediate release, sustained release, controlled release, delayed release, timed release, extended release, or any combination thereof.

14. The drug delivery method of claim 1, wherein the administration of the pharmaceutically active agent is continuous delivery.

15. The drug delivery method of claim 1, the administration of the pharmaceutically active agent is pulsatile delivery.

16. The drug delivery method of claim 1, which comprises: at least two doses of pharmaceutically active agent administered during at least one 24 hour period of time to provide effective therapeutic levels of the pharmaceutically active agent at a site or sites of action in human or an animal over said period, wherein the pharmaceutically active agent is administered in uneven doses and over varying time intervals, and wherein the uneven doses and the varying time intervals are selected to optimize levels of the pharmaceutically active agent at the site or sites of action for maximum efficacy and for the treatment of diseases associated with disorders of circadian rhythm or other chronobiological malady

17. A method of treating, preventing, inhibiting, or modulating adverse skin reactions, irritation or sensitization produced by the components of a topical drug formulation of a pharmacologically active agent, by co-administration of 0.1% to 5% of one or more anti-oxidant botanical agents derived from natural products and plant extracts.

18. The method of claim 17, wherein the antioxidants botanical agents derived from natural plant sources, are polyphenols.

19. The method of claim 18, wherein the polyphenols are selected from the group consisting of: hydroxybenzoic acids, their congeners, and their esters, ferulic acid and chlorogenic acid and curcumin; green tea extracts; chalcone [614-47-1], quercetin [117-39-5], genestein [446-72-0] and daidzein [486-66-8] and their congeners and esters, and the powder of cocoa bean plants.

20. The method of claim 17, wherein the antioxidants botanical agents derived from natural plant sources, are phenols.

21. The method of claim 20, wherein the phenols are selected from the group consisting of: flavones, flavonols, isoflavones, and flavonones.

22. The method of claim 20, wherein the phenols are selected from the group consisting of: catachin [154-234*], chalcone [614-47-1], epicatechin [490-46-0] epicatechin gallate [1257-08-5], epigallocatechin [970-74-1], epigallocatechin gallate [989-51-5] and their congeners and esters.

23. A method of treating, preventing, inhibiting, or modulating adverse skin reactions, irritation or sensitization produced by the components of a topical cosmetic formulation comprising an active agents, by co-administration of 0.1% to 5% of one or more anti-oxidant botanical agents derived from natural products and plant extracts.

24. The method of claim 23, wherein the antioxidants botanical agents derived from natural plant sources, are polyphenols.

25. The method of claim 24, wherein the polyphenols are selected from the group consisting of: hydroxybenzoic acids, their congeners, and their esters, ferulic acid and chlorogenic acid and curcumin; green tea extracts; chalcone [614-47-1], quercetin [117-39-5], genestein [446-72-0] and daidzein [486-66-8] and their congeners and esters, and the powder of cocoa bean plants.

26. The method of claim 23, wherein the antioxidants botanical agents derived from natural plant sources, are phenols.

27. The method of claim 26, wherein the phenols are selected from the group consisting of: flavones, flavonols, isoflavones, and flavonones.

28. The method of claim 26, wherein the phenols are selected from the group consisting of: catachin [154-23-4*], chalcone [614-47-1], epicatechin [490-46-0] epicatechin gallate [1257-08-5], epigallocatechin [970-74-1], epigallocatechin gallate [989-51-5] and their congeners and esters.
